(19) United States
(12) Patent Application Publication
(10) Pub. No.: US 2007/0282388 A1
(43) Pub. Date: Dec. 6, 2007

(54) Method and apparatus for treatment of amyotrophic lateral sclerosis patients

(76) Inventor: Reuven Sandyk, Roslyn, NY (US)

Correspondence Address: Dennis M. Flaherty, Esq.;Ostrager Chong Flaherty & Broitman P.C., Suite 825, 250 Park Avenue, New York, NY, 10177-0899, US

(21) Appl. No.: 11/444,605

(22) Filed: Jun. 1, 2006

(51) Int. Cl. A61N 1/00

(52) U.S. Cl. 607/45

(57) ABSTRACT

A method for treating patients with Amyotrophic Lateral Sclerosis (ALS), also known as Lou Gehrig's disease. In accordance with the method of treatment, transcranial AC pulsed electromagnetic fields (EMFs) in substantially square waveforms in particular frequency and amplitude ranges and picotesla quantities are applied to the patient's brain. EMFs are applied via a transducer array containing a plurality of flexible circular coils in an arrangement placed over the scalp of an ALS patient. The treatment method ameliorates symptoms associated with the disease state and slows the progression of the disease.

### BACKGROUND OF THE INVENTION

The present invention relates to a method and system for the treatment of patients with Amyotrophic Lateral Sclerosis (ALS), also known as Lou Gehrig's disease.

Amyotrophic lateral sclerosis (ALS), more commonly known as Lou Gehrig's disease, is a progressive invariably fatal neuromuscular disorder characterized clinically by weakness, spasticity, muscle wasting, and weight loss that results in death in 50% of patients within three years of diagnosis. Approximately 30,000 Americans currently are diagnosed with ALS and 5,000 are diagnosed each year with the disease, and of these 10% are under 40 years of age. About 5% to 10% of ALS cases are hereditary. ALS is the third most common neurodegenerative cause of adult death, after Alzheimer's disease and Parkinson's disease.

ALS has been recognized as an important clinical syndrome since the middle of the 19th century. The French neurologist, Jean-Martin Charcot first identified in 1869 that motoneurons from the spinal cord are the main part of the nervous system affected by this disease. Presently, there is no specific biological marker for ALS and the diagnosis depends upon the recognition of a characteristic clinical constellation that includes the presence of both upper and lower motor neuron findings and progressive motor dysfunction. These are supported by electrophysiological findings such as needle electromyography (EMG) and nerve conduction studies. A middle-aged patient who presents with a combination of painless and progressive, asymmetrical muscle weakness with wasting, fasciculations and cramps, in a multimyotomal distribution that is associated with upper motor neuron signs, a normal sensory examination, and normal sphincter and ocular function, almost always has ALS. Patients initially present with symptoms in an arm (40%-60%), such as weakness or difficulty with dexterity, or in a leg (20%), such as tripping or foot dragging. Difficulty in speech or swallowing is the initial symptom in the bulbar form of the disease which affects approximately 20%-25% of patients. Overt clinical dementia occurs in less than 5% of ALS patients but, using formal psychometric testing, as many as 35% of patients show some evidence of cognitive impairment. Prior to onset of overt symptoms, some patients experience subtle clinical features, including cramps, muscle fasciculations as well as heat and exercise intolerance. Approximately 50% to 80% of spinal ventral horn cells can be lost before weakness or muscle wasting occurs. Over the course of the disease, patients develop severe, progressive muscle weakness often requiring ventilatory support and gastrostomy. Death usually is attributed to respiratory failure or cachexia.

Selective degeneration of motoneurons is the pathological hallmark of ALS. Histologically, there is decay of motor endplates, axonal degeneration, and loss of motoneurons. It is estimated that in ALS 70% of functional spinal alpha-motoneurons are lost in the first post-onset year. Large motoneurons of the ventral horns of the spinal cord and motor nuclei of the brainstem (V motor, VII motor, IX and X somatic motor, and XII motor) as well as large pyramidal neurons of the motor cortex and/or large myelinated axons of the corticospinal tracts are affected by the disease. The motoneurons of cranial nerves III, IV and VI (which innervate extraocular muscles), the intermediolateral column of the spinal cord, and sacral spinal cord motoneurons (S-2) of the nucleus of Onufrowicz (which innervates the external sphincter muscle of the urethra and anus) are usually unaffected in the disease. The disease is confined to the voluntary motor system and even patients with an advanced stage of the disease usually show a remarkable absence of sensory, intellectual, cerebellar, and extrapyramidal involvement.

The mechanisms and processes responsible for the selective loss of motoneurons in ALS patients remain unknown. It has been suggested that susceptibility to the disease is acquired early in life. ALS mortality was associated with rural farming and socioeconomic status but not with urbanization, physician-population ratios, lead or mercury exposure, or cancer. However several hypotheses have been put forward, including oxidative damage and/or toxicity from intracellular aggregates due to mutant superoxide dismutase-1 activity, axonal strangulation from cytoskeletal abnormalities, loss of trophic factor support and glutamate-mediated excitotoxicity.

Currently there is no specific treatment that either effectively ameliorates the symptoms of the disease or slows its progression. Riluzole, the only drug currently approved by the FDA for the treatment of ALS, has shown only modest effects on survival, but has no significant effect on functionality. The invention disclosed below is predicated on the theory that the pineal gland, generally recognized as a photoreceptor as well as a magnetosensor organ, is intimately related to melatonin release and neurochemical cascade effects associated with the production and release of the neurotransmitter serotonin (5-HT). Melatonin is a potent inhibitor of glutamate neurotoxicity and thus exerts a neuroprotective effect by blocking one or more steps of the oxidation cascade which lead to neuronal degeneration. Melatonin also stimulates 5-HT production in specialized neurons in the brainstem raphe nuclei that project to brainstem and spinal motoneurons. The levels of 5-HT and its metabolite 5-HIAA are reduced in the spinal cord of ALS patients. Postmortem tissue from ALS patients also showed a reduction in 5-HT1A receptor binding in the cervical spine and a 20% reduction of 5-HT2 receptor binding across all cortical laminae. 5-HT is a potent modulator of motoneuron excitability in the spinal cord, brainstem, and motor cortex. In the motoneuron 5-HT acts to facilitate glutamate-induced neuronal excitability. Because of its facilitatory effects on glutamatergic motoneuron excitation, 5-HT may be pivotal to the pathogenesis and therapy of ALS. [Sandyk, R., “Serotonergic mechanisms in amyotrophic lateral sclerosis.” International Journal of Neuroscience (in press).] Thus the premise is itself novel, although not a necessary element of the present invention. Indeed, while theoretical, the actual facts demonstrate efficacy, and the theory, would require years of development and comprehension (including recognition and understanding of the brain as an electromagnetic-chemical organ).

There is a need for a method of treatment that effectively ameliorates the symptoms of ALS patients and slows the progression of the disease.

### BRIEF DESCRIPTION OF THE INVENTION

The present invention is an effective method of treatment for improving or ameliorating the symptoms of patients suffering from ALS. The method seeks to trigger the pineal gland via picotesla-flux-density AC-pulsed electromagnetic fields (EMFs) induced from an array of coils placed on the skull of the patient. The treatment method of the instant invention is completely differentiated from the pharmaceutical treatments heretofore employed, in that it utilizes transcranial AC-pulsed applications of EMFs in substantially square waveforms constructed from multiple sinusoidal waves at different frequencies in the picotesla flux density, applied to the patient's brain via a transducer array containing a plurality of spiral coils, to successfully ameliorate (if not eliminate specific symptoms) the symptoms of ALS and slow the progression of the disease.

One aspect of the invention is a method for treating patients diagnosed as having a neurological disorder, comprising the following steps: (a) placing an electromagnetic radiation transmitter in proximity to a head of a patient having a neurological disorder; and (b) activating the electromagnetic radiation transmitter to transmit electromagnetic radiation having a flux density of 100 picotesla or less into the brain of the patient, wherein the activating step comprises the step of supplying an alternating current with a substantially square waveform having a duty cycle of 40 to 60%, a rise time and a fall time of less than 20 microseconds, and a frequency in a range of 2 to 5 Hertz.

Another aspect of the invention is a device for treating patients diagnosed as having a neurological disorder, comprising a waveform generator and an electromagnetic radiation transmitter electrically connected to the waveform generator, wherein the waveform generator is set to cause the electromagnetic radiation transmitter to transmit electromagnetic radiation having a flux density of 100 picotesla or less within a range of a few inches, the settings being such that the waveform generator supplies an alternating current to the electromagnetic radiation transmitter, the alternating current having a substantially square waveform with a duty cycle of 40 to 60%, a rise time and a fall time of less than 20 microseconds, and a frequency in a range of 2 to 5 Hertz.

A further aspect of the invention is a coil array comprising a multiplicity of electrically conductive coils and means for electrically connecting the coils in series, wherein each of the coils comprises a spiral-shaped electrical conductor having a generally central terminal and a generally peripheral terminal, and the electrically connecting means comprise a multiplicity of electrical connectors, each of the electrical connectors connecting a generally central terminal of one coil to a generally peripheral terminal of another coil such that all of the coils are connected in series.

Other aspects of the invention are disclosed and claimed below.

### BRIEF DESCRIPTION OF THE DRAWINGS

FIG. 1 is a block diagram depicting a system for treating ALS patients in accordance with one embodiment of the present invention.

FIG. 2 is a drawing showing a coil array for suitable for skull placement for treatment of a patient in accordance with one embodiment of the invention.

Reference will now be made to the drawings in which similar elements in different drawings bear the same reference numerals.

### DETAILED DESCRIPTION OF THE INVENTION

The present invention is an effective method of treatment for improving or ameliorating the symptoms of patients suffering from ALS. The method seeks to trigger the pineal gland via picotesla-flux-density AC-pulsed electromagnetic fields (EMFs) induced from an array of coils placed on the skull of the patient. The treatment method of the instant invention utilizes transcranial AC-pulsed applications of EMFs in substantially square waveforms constructed from multiple sinusoidal waves at different frequencies in the picotesla flux density, applied to the patient's brain via a transducer array containing a plurality of spiral coils, to successfully ameliorate (if not eliminate specific symptoms) the symptoms of ALS and slow the progression of the disease. It is theorized herein that the pineal gland is pivotal in the pathogenesis of ALS, and that EMF treatment, via transduction through the cranium aimed by a flexible coiled array at the pineal gland, affects 5-HT neurotransmission when picotesla levels at specific, signature waveforms, amplitudes, frequencies, and duty cycles are applied to ameliorate neurological conditions. In particular, the subject invention relates to, but is far and away an improvement over, the studies performed by the subject inventor, including those disclosed and claimed in his other patents including, without limitation, U.S. Pat. Nos. 5,470,846; 5,691,324; 5,691,325; 5,885,976, the contents of which are incorporated by reference herein.

The invention is a method whereby an ALS patient is pulsed with EMFs at picotesla flux density levels. Such EMFs are applied via a transcranial, flexible, skull plate having a plurality of serially connected circular coils in a transducer array. The method involves AC-pulsed application in substantially square waveforms at a frequency range which corresponds to the lower range of theta activity recorded from the skull on the electroencephalogram (EEG) applied to the patient's brain. The effect of the treatment is to ameliorate symptoms of ALS and thereby slow (if not cease) the progression of the disease.

Patients treated with AC pulsed EMFs have experienced dramatic improvements in, inter alia, fatigue, endurance, mood, appetite with weight gain, and sleep (with recurrence of dreams) as well as increased muscle strength and muscle bulk in the limbs resulting in improvement in mobility, manual dexterity, trunk and shoulder control, chewing, swallowing, and breathing. There were also improvements in autonomic functions including bladder and bowel control, sweating, blood pressure and heart rate, and skin temperature. In addition, this treatment appeared to slow the progression of the disease and in the majority of patients the course of the disease was stabilized and these patients showed continued improvement over several months of observation.

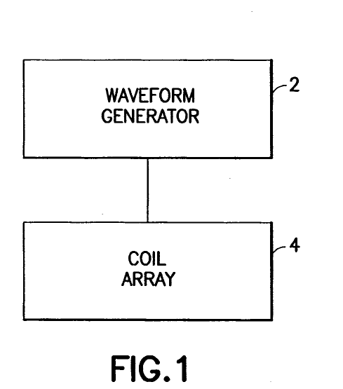

FIG. 1 shows a system comprising a waveform generator 2 and a flexible coil array 4. The coil array is placed on the skull of the patient to be treated, the flexible array being conformed to the shape of the skull. Preferably, the coil array is arranged such that the weak EMFs are directed at the pineal gland. The waveform generator 2 energizes the coil array 4 via an alternating electrical current. For optimal therapeutic effects during treatment, the waveform generator supplies an alternating current having a substantially square waveform with a duty cycle in the range of 40 to 60%, a rise time and a fall time of less than 20 microseconds, and a frequency in the range of 2 to 5 Hertz. The amplitude of the alternating current is in the range of 0.5 to 1.5 microamperes. The coil array is such that, in response to current of the foregoing type, weak electromagnetic fields having a flux density of 10 to 100 picotesla are produced within a range of several inches, i.e., within a range encompassing portions of the brain of the patient being treated when the array is on top of the skull.

In accordance with a preferred treatment protocol, the substantially square waveform is symmetric, has a duty cycle of approximately 50%, and has a frequency of approximately 3.45 Hertz. Preferably the alternating current has an amplitude of 1.2 microamperes.

The substantially square waveforms produced by the waveform generator 2 are constructed from multiple sinusoidal waves at different frequencies. The waveform generator 2 comprises a current generator and an output resistor, by which the current generator is coupled to the coils. The waveform generator 2 also includes controls for selecting the frequency and duty cycle of the alternating current, the waveform of the current, and the amplitude of the current.

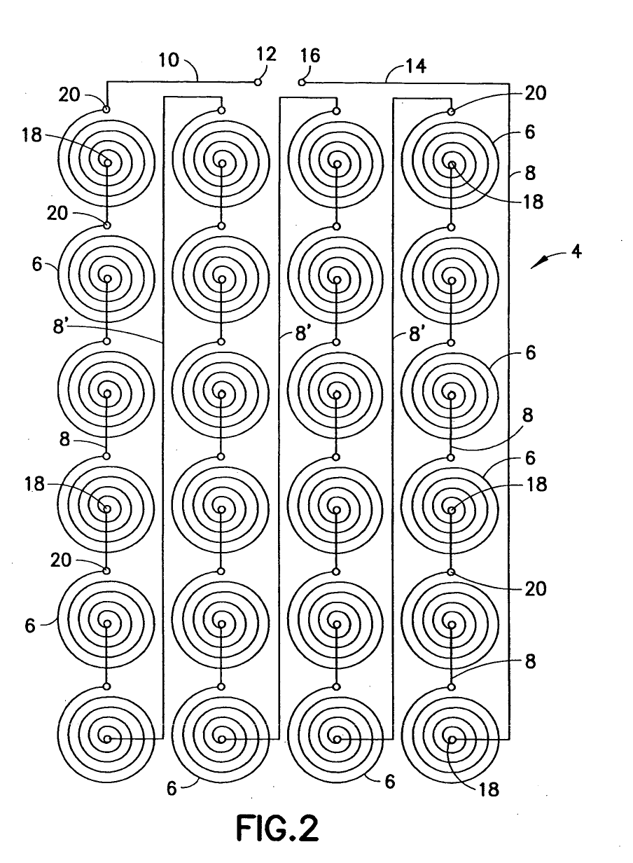

Referring to FIG. 2, the coil array 4 comprises a multiplicity of electrically conductive spiral-shaped coils 6 arranged in rows and columns. In the implementation shown in FIG. 2, the array has 24 spiral coils. The coil array 4 further comprises means for electrically connecting the coils in series. Each coil 6 comprises a spiral-shaped electrical conductor having a generally central terminal 18 and a generally peripheral terminal 20. The coils are electrically connected in series by means of a multiplicity of electrical connectors 8 and 8′. Each electrical connector 8 connects a generally central terminal 18 of one coil in a column with a generally peripheral terminal 20 of another coil in the same column such that all of the coils in each column are connected in series. Each electrical connector 8′ connects a generally central terminal 18 of the lowermost coil in one column with a generally peripheral terminal 20 of the uppermost coil in an adjacent column such that the columns of coils are connected in series. In addition, the coil array comprises a pair of array terminals 12 and 16, by means of which the coil array can be electrically connected to the corresponding output terminals of the waveform generator. The array terminal 12 is electrically connected to the generally peripheral terminal 20 of the first of the series of spiral coils by means of an electrical conductor 10, whereas the array terminal 16 is electrically connected to the generally central terminal 18 of the last of the series of spiral coils by means of an electrical conductor 14. Preferably, the coil array is attached to a flexible substrate. The flexible substrate is held on top of the patient's skull by a headgear assembly (not shown in the drawings).

The medical administrator performs a method for treating an ALS patient comprising the following steps: (a) placing the coil array in proximity to the head of a patient that has been diagnosed with ALS; and (b) activating the coil array to transmit electromagnetic radiation having a flux density of 100 picotesla or less into the brain of the patient, wherein the activating step comprises the step of supplying an alternating current with a substantially square waveform having a duty cycle of 40 to 60%, a rise time and a fall time of less than 20 microseconds, a frequency in a range of 2 to 5 Hertz. The alternating current has an amplitude in the range of 0.5 to 1.5 microamperes.

During application of AC-pulsed EMFs having a flux density in the range of 10 to 100 picotesla, patients experienced regularly intermittent sensations of itching, tingling, twitching, and sneezing and also a sense of pulsations in the affected muscle groups coupled with frequent episodes of yawning and stretching. In contrast to other neurological disorders treated with AC-pulsed EMFs (e.g. multiple sclerosis, Parkinson's disease, Alzheimer's disease), treatment using the instant method does not result in a decline in function between the treatment sessions and sensory phenomena such as itching, tingling, twitching, and sense of pulsations in various muscle groups continuing for hours and even days after the termination of EMF treatment. Also in contrast to other neurological disorders treated with AC-pulsed EMFs, treatment using the instant method, ALS patients responded to a specific EMF signal characteristic, irrespective of the presenting clinical symptoms, course of the disease, or duration of the disease.

The signal characteristics include the waveform, rise and fall times of the square wave, amplitude, frequency, and duty cycle of the EMF radiation. Even a small deviation from the patient's signal characteristics utilized in the instant treatment method can result in a lack of response or potentially rapid worsening of symptoms (e.g., weakness of the legs and hands, difficulties swallowing and breathing, etc.). The substantially square wave used in the instant treatment method is believed to be the critical and fundamental waveform for the treatment of ALS. A substantially square wave is mathematically equivalent to the sum of a sinusoidal wave at that same frequency, plus an infinite series of odd-multiple frequency sinusoidal waves at diminishing amplitudes. The substantially square wave has large harmonics at odd multiples of the fundamental frequency (e.g., odd integer harmonics). An ideal square wave is composed of an infinite number of odd harmonics. Before filtering, the sinusoidal waves are in phase. The effects of a substantially square wave on the symptoms and course of ALS are suspected to be related to a sum of multiple sinusoidal waves of various frequencies and phases interacting with the pineal gland and possibly also directly with cortical motoneurons. The duty cycle of the simulating signal ranges from 40% to 60%. The rise and fall times are equal in duration (<20 microseconds). The optimal frequency of the signal is 3.45 Hertz with a period of 0.29 second, which corresponds to the low theta (electroencephalogram) EEG activity (3-7 Hertz) recorded from the human skull with surface electrodes. The optimal amplitude (0.5 to 1.5 microamperes) of the current signal produces a magnetic field flux density of 10 to 100 picotesla, which is within the range of the magnetic field generated by neural activity in the human brain.

### Case History of an ALS Patient Treated with AC-Pulsed EMFs

A 48-year-old left-handed male computer programmer developed weakness of the right hand in 2000 with difficulty holding objects and frequently dropping objects with his right hand. In 2001, he noticed his right hand was clumsy while manipulating a computer mouse. At the end of 2001, he developed difficulty writing with his left hand. In January of 2002, electromyography (EMG) was performed and showed acute and chronic denervations in the upper extremities, including cervical, thoracic and lumbar paraspinal muscles. In March 2002, he was diagnosed with amyotrophic lateral sclerosis (ALS) at Columbia-Presbyterian Medical Center.

In April 2002, he developed muscle cramps in his hands at rest as well as on exertion. EMG performed in May 2002, involving evaluation of selected muscles in the right leg and both upper extremities, revealed fibrillations and fasciculations, polyphasic, large-amplitude motor units and reduced recruitment on maximal effort. EMG examination of the tongue revealed fibrillations and full recruitment, indicating progression of the disease.

Over the following 3 years the patient developed increasing weakness of his arms, hands and fingers. Simultaneously, he also experienced progressive weakness of the legs resulting in bilateral foot drop. There was also weakness of the trunk musculature and he had difficulties sitting upright. He became wheelchair dependent in 2003 and required foot drop braces. He denied difficulties with speech or swallowing. About one year ago he experienced occasional shortness of breath with stress. He fatigued easily and yawned frequently during the day. He reported to sleep about 10 hours per night and recalled frequent dreaming (none in color). The fatigue was not relieved by sleep and was particularly severe in the morning until about 1 p.m. He was treated with modafinil for the past year with little improvement in fatigue. He denied cognitive difficulties but he experienced some slowing of thought processes. He weighed 165 pounds as compared to 190 pounds 3 years ago. His appetite was fair and he experienced daily episodes of nausea in the morning. He also experienced heat intolerance. He had increased daytime urinary frequency, voiding almost hourly with nocturia X1. He carried a urinal with him when traveling outside his home. He denied pain or visual problems. He had intermittent cramping in the legs. He denied experiencing spontaneous itching, tingling or pulsations in the extremities. His medications included riluzole 50 mg twice daily, modafinil 100 mg daily, and sertraline 50 mg daily.

On examination on Aug. 16, 2005 his blood pressure was 110/70 mmg, pulse was 78/min and regular, and p02 was 93% on pulse oximetry. He was alert and fully oriented without speech impairment. There was no pseudobulbar affect but he appeared depressed. Examination of the cranial nerves was unremarkable. Motor examination revealed profound weakness in both upper and lower limbs (upper extremities: biceps 2+/5 left, 2/5 right; triceps 2+/5 left, 2/5 right; wrist extension 2+/5 left, 2/5 right; finger abduction 1+/5 left, 1/5 right; lower extremities: hip flexion 2+/5 left, 1+/5 right; knee extension 3+/5 left, 2+/5 right; hip adduction 2+/5 left, 1+/5 right; foot inversion 2+/5 left, 1+/5 right). There was flexion of the middle digits of both hands. He was unable to hold objects in his hands or grasp small objects with his fingers due to limited range of movement of the thumbs and forefingers. There was marked atrophy of the muscles of the upper arms and shoulders (particularly the deltoids), forearms, and intrinsic hand muscles. There was moderate atrophy of the back musculature. There were diffuse fasciculations in the upper limbs, chest, back musculature, and lower limbs. Biceps and triceps tendon reflexes were 1+ bilaterally. Hoffmann's sign was negative bilaterally. Knee jerks and ankle reflexes were 2+ bilaterally. Plantar responses were flexor bilaterally. Muscle tone was increased in the legs, greater on the right. He wore braces on both ankles. He was unable to elevate his legs to walk. He had a broad-based waddling gait. He fatigued after walking about 10 feet and promptly requested to sit back in the wheelchair. There was no sensory loss or cerebellar signs.

### Treatment with AC-Pulsed Electromagnetic Fields

Pulsed electromagnetic fields (EMFs) were administered transcranially over the patient's scalp for 9 months at a rate of 3 treatments per week. Each treatment was administered for 2 hours, usually between 1 and 3 p.m. The EMFs were applied via a transducer coil array in a dark room that was magnetically unshielded.

The frequency of the AC-pulsed EMF was 3.45 Hertz for 6 months and 4.75 Hertz for the past 3 months. The amplitude of the current supplied to the coil array was 1.20 microamperes. The waveform was square (rectangular) with a duty cycle of 50% and rise and fall times of less than 20 microseconds.

### Response to Treatment with AC-Pulsed Electromagnetic Fields

Within the first hour, the patient developed recurrent episodes of yawning (12 times during 60 minutes). There were itching sensations in the face (left side), forehead and nose. There was increased strength in the lower limbs (able to raise both legs higher, particularly left leg) and trunk (sitting more erect). There was increased range of finger movements including the thumbs. Fingers felt less stiff and he was able to open and close his hands with greater ease. Mentally, patient felt a surge in energy and mood elevation. During the second hour he continued to yawn frequently (13 times during 60 minutes). The itching sensations in the face, forehead and nose were more intense. He felt tingling sensations in the fingertips. Towards the end of treatment, he felt hungry and craved for sweets.

Two days later, the patient returned for treatment. He reported experiencing a constant urge to yawn. In addition, he experienced increased sweating and markedly reduced need to urinate (from hourly prior to treatment to now every 2-3 hours). Also, for two prior days he woke up 2 hours earlier in the morning and reported feeling more energetic. Functionally, he was able to rise from the chair without assistance. His legs and trunk felt stronger. Blood pressure was 110/70 mm Hg, pulse 85/min and p02 was 97% on pulse oximetry. His mood was optimistic and he was smiling. He walked a longer distance (20 feet) unassisted at a faster pace. Finger abduction was 2+/5 on the left and 2/5 on the right. Hip flexion was 3+/5 on the left and 2+/5 on the right.

He subsequently received his second electromagnetic treatment of 2 hours duration, during which time he yawned 45 times (deep and prolonged yawns). Yawning was often associated with stretching of the trunk and arms. During treatment, the patient experienced itching sensations in the face, eyes, forehead and nose, tingling sensations in the fingertips and thumbs, twitching sensations in the back and shoulders, and pulsations in the arms and legs. He felt warm and his nose felt slightly congested. The patient sneezed twice successively. These sensations did not occur spontaneously prior to initiation of electromagnetic treatment. Mentally, he felt more alert and noted increased strength in the lower limbs (he was able to raise both legs significantly higher, particularly his left leg). His left shoulder became more mobile and his abdominal musculature felt tighter.

During 9 months of electromagnetic treatment, the patient experienced a host of radical changes in his disease. He experienced a sense of well being, which, according to the patient, “I have not felt in many months.” His level of energy increased dramatically throughout the course of the day. He had no fatigue. He slept well throughout the night and recalled dreaming, frequently in color. He woke up spontaneously at about 7:30 a.m. without fatigue. The nausea and dizziness sensations abated. He experienced improved heat tolerance and was able to sit in the sun without experiencing nausea. Frequency of urination declined dramatically and he voided 3 times daily. He abandoned the use of a urinal. Appetite improved and he gained about 12 pounds in 9 months. He experienced a sense of mental clarity and his wife reported that he became more assertive in conversations. On several occasions he slept on his left side without experiencing any shoulder pain in the morning.

He became more functional in daily activities. He frequently arose from bed unassisted. He felt more stable on his feet. He stood significantly longer before experiencing weakness in his knees and legs. During meals, he brought food up to his mouth with his left arm instead of leaning down to reach for food. There was improvement in pincer movements of the left hand and increased control and range of movement of the thumbs. He was able to brush his hair with the left hand and dial the telephone due to improved hand strength. He crossed his legs with no effort. On one occasion he recalled visiting a book store and walking around for about 45 minutes, pushing his wheelchair in front of him. Unassisted, he walked about 50 feet before needing to rest.

On Apr. 16, 2006, the patient stated that he now functioned at the level of at least one year ago. Motor examination revealed increases in both upper and lower limbs (upper extremities: biceps 3/5 left, 2+/5 right; triceps 3/5 left, 2+/5 right; wrist extension 3/5 left, 2+/5 right; finger abduction 2+/5 left, 2/5 right; lower extremities: hip flexion 3+/5 left, 2+/5 right; knee extension 3+/5 left, 3/5 right; hip adduction 3+/5 left, 2+/5 right; foot inversion 3+/5 left, 2+/5 right).

While the invention has been described with reference to particular embodiments, it will be understood by those skilled in the art that various changes may be made and equivalents may be substituted for members thereof without departing from the scope of the invention. In addition, many modifications may be made to adapt a particular situation to the teachings of the invention without departing from the essential scope thereof. Therefore it is intended that the invention not be limited to the particular embodiment disclosed as the best mode contemplated for carrying out this invention, but that the invention will include all embodiments falling within the scope of the appended claims.

1. A method for treating patients diagnosed as having a neurological disorder, comprising the following steps:
(a) placing an electromagnetic radiation transmitter in proximity to a head of a patient having a neurological disorder; and
(b) activating said electromagnetic radiation transmitter to transmit electromagnetic radiation having a flux density of 100 picotesla or less into the brain of said patient,
wherein said activating step comprises the step of supplying an alternating current with a substantially square waveform having a duty cycle of 40 to 60%, a rise time and a fall time of less than 20 microseconds, and a frequency in a range of 2 to 5 Hertz.

2. The method as recited in claim 1, further comprising the step of diagnosing said patient as having amyotrophic lateral sclerosis.

3. The method as recited in claim 1, wherein the amplitude of said alternating current is in the range of 0.5 to 1.5 microamperes.

4. The method as recited in claim 1, wherein said electromagnetic radiation transmitter comprises an array of spiral coils connected in series.

5. The method as recited in claim 3, wherein the number of coils is 24.

6. The method as recited in claim 1, wherein the substantially square waveform is symmetric.

7. The method as recited in claim 1, wherein the frequency of said alternating current is approximately 3.45 Hertz.

8. The method as recited in claim 1, wherein the amplitude of said alternating current is approximately 1.2 microamperes.

9. The method as recited in claim 1, wherein the duty cycle of said alternating current is approximately 50%.

10. The method as recited in claim 1, wherein the transmitted electromagnetic radiation is directed to the region of the pineal gland.

11. A device for treating patients diagnosed as having a neurological disorder, comprising a waveform generator and an electromagnetic radiation transmitter electrically connected to said waveform generator, wherein said waveform generator is set to cause said electromagnetic radiation transmitter to transmit electromagnetic radiation having a flux density of 100 picotesla or less within a range of a few inches, said settings being such that said waveform generator supplies an alternating current to said electromagnetic radiation transmitter, said alternating current having a substantially square waveform with a duty cycle of 40 to 60%, a rise time and a fall time of less than 20 microseconds, and a frequency in a range of 2 to 5 Hertz.

12. The device as recited in claim 11, wherein the amplitude of said alternating current is in the range of 0.5 to 1.5 microamperes.

13. The device as recited in claim 11, wherein said electromagnetic radiation transmitter comprises an array of spiral coils connected in series.

14. The device as recited in claim 13, wherein the number of coils in said array is 24.

15. A coil array comprising a multiplicity of electrically conductive coils and means for electrically connecting said coils in series, wherein each of said coils comprises a spiral-shaped electrical conductor having a generally central terminal and a generally peripheral terminal, and said electrically connecting means comprise a multiplicity of electrical connectors, each of said electrical connectors connecting a generally central terminal of one coil to a generally peripheral terminal of another coil such that all of said coils are connected in series.

16. The coil array as recited in claim 15, further comprising first and second array terminals, means for electrically connecting said first array terminal to a generally central terminal of a first one of said coils, and means for electrically connecting said second array terminal to a generally peripheral of a last one of said coils.

17. The coil array as recited in claim 15, wherein the number of coils in said array is 24.

18. The coil array as recited in claim 17, wherein the coils are arranged in six rows and four columns.
